(19) United States
(12) Patent Application Publication
(10) Pub. No.: US 2007/0276211 A1
(43) Pub. Date: Nov. 29, 2007

(54) COMPACT MINIMALLY INVASIVE BIOMEDICAL MONITOR

(76) Inventors: Jose Mir, Rochester, NY (US); James Kelly Lee, Rochester, NY (US)

Correspondence Address: JAECKLE FLEISCHMANN & MUGEL, LLP, 190 Linden Oaks, ROCHESTER, NY, 14625-2812, US

(21) Appl. No.: 11/754,987

(22) Filed: May 29, 2007

Related U.S. Application Data
(60) Provisional application No. 60/803,289, filed on May 26, 2006.

(51) Int. Cl. A61M 31/00; A61B 5/05

(52) U.S. Cl. 600/345; 604/65

(57) ABSTRACT

A biomedical monitor is disclosed. The biomedical monitor has an array of moveable microneedles coated with a first chemical sensing media. The biomedical monitor also has an actuator configured to move at least one microneedle in the array of microneedles from a retracted position to an engaged position whereby the at least one microneedle enters a subject's skin. The biomedical monitor further has an optical system configured to illuminate the at least one microneedle during or after entering the subject's skin and monitor the first chemical sensing media from the at least one microneedle, whereby at least one biomedical characteristic is determined based on at least one spectral property of the monitored first chemical sensing media. A method of monitoring at least one biomedical characteristic is also disclosed.

### RELATED APPLICATIONS

This patent application claims priority to U.S. provisional patent application 60/803,289 entitled “Compact Minimally Invasive BioMedical Monitor,” which was filed May 26, 2006. The 60/803,289 patent application is hereby incorporated by reference in its entirety.

### FIELD

The claimed invention relates to biomedical monitors, and more specifically to compact minimally invasive biomedical monitors.

### BACKGROUND

Existing methods to measure blood glucose suffer from a number of disadvantages. The well-known fingerstick monitor requires the use of a fine lancet that pierces the skin and is able to draw blood for subsequent measurement. Unfortunately, as a result of the discomfort and inconvenience of the process, compliance tends to be low, especially for younger (active) and older patients. Repeated piercing can also lead to sensitivity and/or hardening of the subject's skin since fingertips are one of the body's most sensitive regions. Furthermore, fingerstick-based monitors only provide a sampled measurement of the subject's blood chemistry even though glucose levels fluctuate rapidly after meals. This creates problems especially for diabetics who need to monitor their glucose levels over 5 times a day, exacerbating usage issues for the patient. It would be desirable to have a more continuous monitoring process that is fully automated, requiring little or no periodic calibration that is less invasive to the patient.

Microneedle technology provides a useful minimally-invasive method to sample blood. Due to their small size, microneedles can pierce skin and sample minute quantities of blood or interstitial fluid with minimal impact and/or pain to the subject. In spite of their advantages, microneedle systems described in the prior art are still somewhat invasive since they extract blood from the patient for the measurement. Implanted in vivo sensors provide another means to sample blood chemistry that do not require blood extraction. Unfortunately, long term use of in vivo sensors or microneedles inserted into subjects is hampered by a process known as “bio-fouling”. Bio-fouling refers to changes in device characteristics caused by its interaction with the in vivo environment as a result of the device's presence. At best, bio-fouling requires frequent calibration to compensate for these changes; more often than not these changes are irreversible and require device replacement.

It would be desirable to achieve a less invasive approach to biomedical monitoring that does not extract blood from the patient, provides longer useful life than in vivo devices, and requires little or no calibration.

### SUMMARY

A biomedical monitor is disclosed. The biomedical monitor has an array of moveable microneedles coated with a first chemical sensing media. The biomedical monitor also has an actuator configured to move at least one microneedle in the array of microneedles from a retracted position to an engaged position whereby the at least one microneedle enters a subject's skin. The biomedical monitor further has an optical system configured to illuminate the at least one microneedle during or after entering the subject's skin and monitor the first chemical sensing media from the at least one microneedle, whereby at least one biomedical characteristic is determined based on at least one spectral property of the monitored first chemical sensing media.

A replaceable array of moveable microneedles is also disclosed. The replaceable array of microneedles has a plurality of microneedles coated with at least one chemical sensing media. The replaceable array of microneedles also has a substrate defining wells to house the microneedles. The replaceable array of microneedles further has at least one restoring spring element coupled between each microneedle and the substrate such that each microneedle is held at least partially in an associated well.

A method of monitoring at least one biomedical characteristic is disclosed. A first microneedle coated with a first chemical sensing media is engaged into a subject's skin. The first chemical sensing media is illuminated. One or more spectral characteristics of light reflected from the first chemical sensing media are monitored. At least one biomedical characteristic is determined based on the one or more spectral characteristics of light reflected from the first chemical sensing media.

### BRIEF DESCRIPTION OF THE DRAWINGS

FIG. 1A illustrates one embodiment of a single microneedle device.

FIG. 1B is a magnified view of one embodiment of a single microneedle device.

FIG. 2A illustrates one embodiment of a microneedle array having multiple microneedles such as the one illustrated in FIG. 1.

FIG. 2B illustrates the reverse side of the embodied microneedle array of FIG. 2.

FIG. 3A schematically illustrates an embodiment of a biomedical monitor prior to testing.

FIG. 3B schematically illustrates an embodiment of a biomedical monitor during testing.

FIG. 4 illustrates an exploded view of one embodiment of a biomedical monitor.

FIG. 5. illustrates a side view of another embodiment of a biomedical monitor.

FIG. 6 illustrates an exploded view of another embodiment of a biomedical monitor.

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

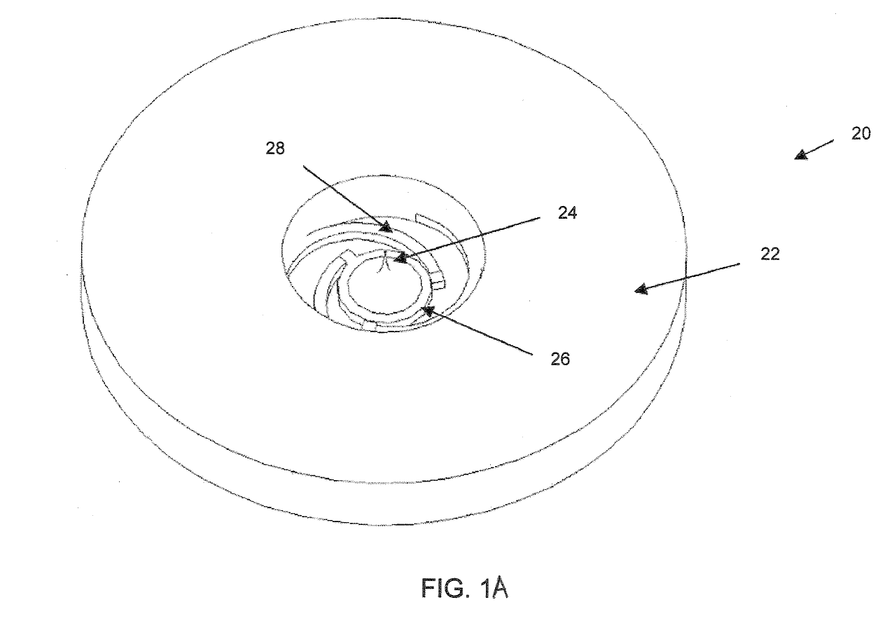

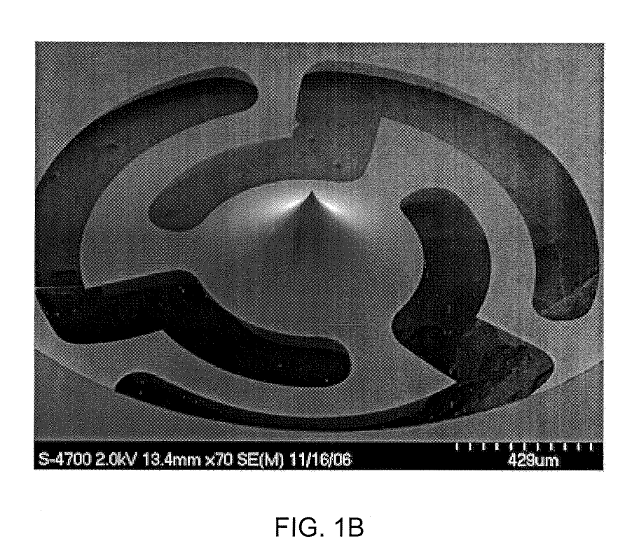

FIG. 1A illustrates one embodiment of a top view of a single microneedle device 20. A substrate 22 has been micromachined to produce a microneedle element 24, supported on microneedle base 26, and at least one restoring spring element 28. Microneedle element 24 should have dimensions such that it is of sufficient length to penetrate the subject's stratum corneum and reach the underlying interstitial fluid or capillary network, e.g. 20-2000 microns, however, in other embodiments, smaller or larger microneedles may be used. Restoring spring element 28 could be patterned directly out of substrate material 22 or out of a layer having desirable mechanical properties that has been deposited onto substrate 22. Alternatively, restoring spring 28 may also be patterned out of one or more materials in a multi-material substrate where additional materials have been deposited or bonded to the substrate 22. For example, an oxidized substrate may be etched to form a microneedle 24 out of silicon and a restoring spring 28 out of either the silicon dioxide layer or a combination of the silicon dioxide layer and the silicon layer. Although not illustrated in this embodiment, other embodiments may include positional sensors on the restoring springs 28 for use in determining the deflection of the microneedle 24. Restoring spring element 28 can be patterned in a number of geometries such as spiral spring (as shown), cantilever structures or other geometries as long as they provide the freedom of movement that allows microneedle element 24 to protrude far enough out of the plane defined by substrate 22 in order to penetrate a subject's skin to a desired depth. A number of substrate 22 and/or microneedle 24 materials maybe used, e.g. silicon, silicon dioxide, silicon nitride, all commonly used in microfabrication or, in general, dielectrics, plastics, metals, glass, or quartz. The microneedle 24 and the microneedle base 26 are preferably transparent, but may be translucent in some embodiments. Several fabrication techniques for the microneedle device 20 are disclosed in the literature, such as photolithography, reactive ion etching, isotropic etching (e.g. for glass), plastic molding, water jet milling, and others may be used. Although hollow microneedles are typically used for drug delivery and diagnostics applications, 24 may be solid, although in some cases, hollow ones may be utilized. Although the embodied microneedle 24 is illustrated as being solid with a smoothly varying cross-section, other embodiments of microneedles may have a constant cross-section. Still other embodiments of microneedles may take on a variety of cross-sectional shapes, including, but not limited to square, circular, triangular, and grooved. Other embodiments of microneedles may be hollow or even corrugated.

FIG. 1B is a magnified view of an actual embodiment of a microneedle device 20 which is this case was formed from quartz.

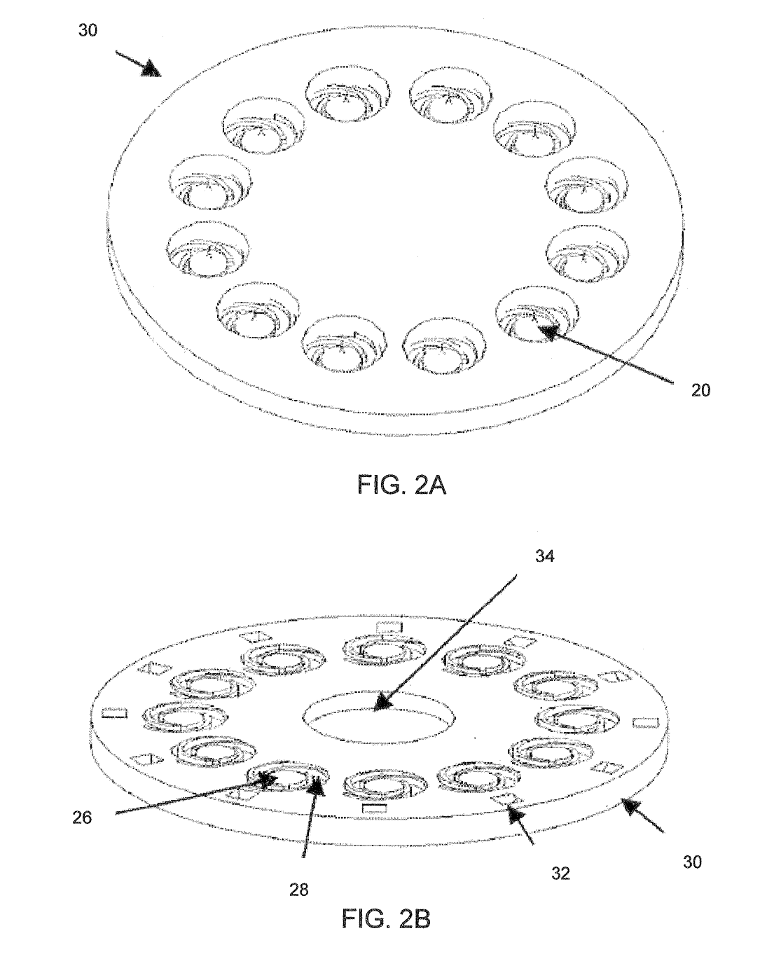

FIG. 2A illustrates one embodiment of an array configuration 30 for the single microneedle device 20 and its supporting elements. The surface in view represents the needle-up side of the device which would normally come in contact with a subject being monitored. Other embodiments may include a film over the microneedles in the microneedle array to prevent the microneedles and the sensing media on the microneedles from interacting with a subject prior to engagement of a particular microneedle. In the example shown, the array of microneedles 30 is patterned radially, although other geometrical arrangements are possible.

FIG. 2B illustrates the needle-down side of the embodiment of FIG. 2A, exposing microneedle bases 26 and restoring spring elements 28. Additional elements may be patterned on the needle-down side of microneedle array configuration 30 such as positional encoder slots 32. Slots 32 are aligned relative to microneedle elements 20, but may be placed at integral number ratios relative to the microneedles, e.g. 1:1, 1:2, 1:3, etc. In this nomenclature, a 1:1 ratio refers to an array having an equal number of microneedles and slots, whereas a 1:3 ratio refers to an array having three times as many microneedles as slots. An optional cylindrical alignment slot 34 may be defined on 30 that is concentric to the circle defined by the array of microneedle devices and their associated positional encoder slots 32.

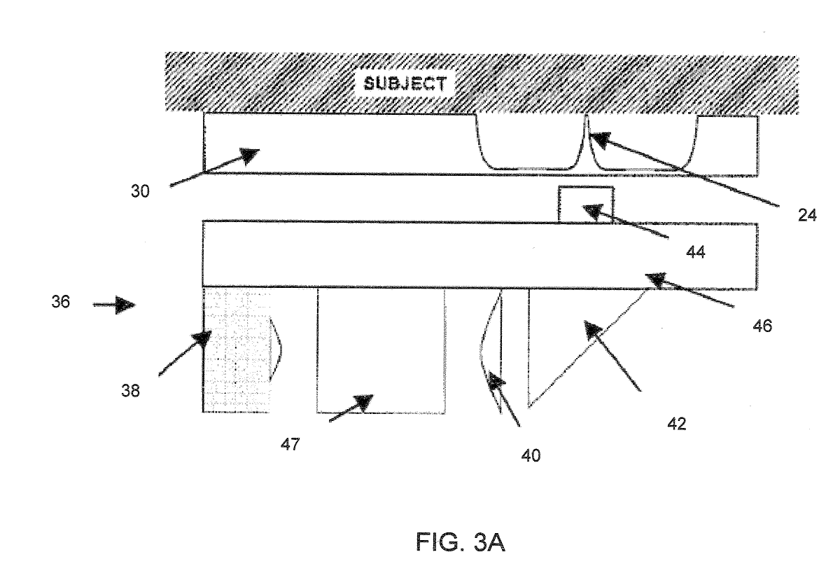

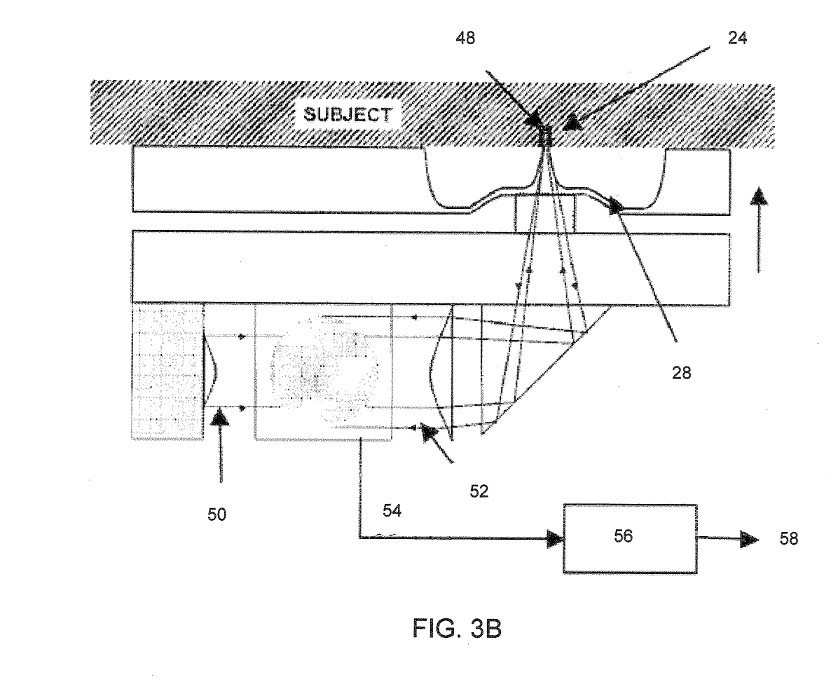

A possible embodiment of array configuration 30 used as a diagnostic device or monitor is schematically illustrated in FIGS. 3A and 3B. FIG. 3A shows a cross-section of microneedle element 24 in its inactivated state positioned within array configuration 30. Microneedle 24 is preferably transparent, but may be translucent. Microneedle element 24 can be coated with chemical sensing material 48 that either changes its color or fluoresces when in contact with a specific chemical specie. For example, sensing material 48 for blood glucose monitoring may use a large number of known glucose sensitive chemicals, e.g. glucose oxidase, glucose dehydrogenase, hexokinase-glucokinase, rhenium bipyridine, boronic acid containing fluorophores, NBD-fluorophores, or any other materials that exhibit the desired chemical and optical response. It should be apparent to those skilled in the chemical arts that these examples of chemical sensing materials are merely illustrative of broader families of chemicals. It will be apparent to those skilled in the chemical arts that the example materials may be modified while still performing the same or similar function of providing or facilitating a spectral response in the presence of a target chemical or chemical compound. All such modifications and equivalents to the listed chemical sensing media as well as alternates for other target media are intended to be included in this disclosure. In some cases, the reagent or fluorophore may need to be incorporated into a polymeric matrix in order to achieve coatability, adhesion, or chemical stability. Other reagents or fluorophores may be used to monitor cholesterol, HDL cholesterol, alcohol, estrogen-progesterone, cortisol, and other physiological chemicals of interest.

FIG. 3A also illustrates an embodiment of an optical system 36 having an electronic light source or light emitter 38, imaging lens 40, reflector 42, and transparent depressor element 44, all of them mounted on transparent actuator substrate 46. Although it is preferred that depressor 44 and actuator substrate 46 are transparent, in some embodiments one or both elements may optionally be translucent. FIG. 3B shows the activated or engaged state of the monitor, achieved when the optical system is pushed against the microneedle array 30. This movement causes the transparent depressor 44 to exert a force on 24 so as to bend restoring spring assembly 28 and achieve penetration of microneedle element 24 into the subject. After the microneedle 24 penetrates the subject, sensing material 48 undergoes a change in color or exhibits fluorescence which is sampled using light beam 50 emanating from light emitter 38. Light emitter 38 could be an incandescent source with collimation optics, a light emitting diode, or a laser diode, for example. The spectral requirements for imaging lens 40 will depend on the wavelength required to monitor absorption of the reagent or excite fluorescence in sensing material 48. Imaging lens 40 focuses light beam 50 to optically sample sensing material 48 as it changes color. Signal beam 52 emanating from sensing material 48 contains information regarding the color change of sensing material 48, is captured by imaging lens 40, and directed toward a light detector 47 via beam splitter (not shown). The light detector 47 may be made selective to the optical absorption or fluorescence wavelengths of sensing material 48. Output from light detector 54 is processed by processor 56 to produce digital data signal 58 representative of the concentration of chemical being monitored. After the measurement is made and data 58 is captured, the optical sensor assembly is withdrawn away from the subject, returning the entire assembly to the configuration shown in FIG. 3A.

Although FIGS. 3A and 3B depict a useful system configuration for the diagnostic device, it should be apparent to those skilled in the art that other system configurations are possible. For example, reflector 42 and/or beam splitter may be omitted or varied if beams 50 and 52 follow trajectories perpendicular to 30. Some embodiments may utilize an off-axis light source so that diffuse light reflected from sensing material 48 may be captured by an image sensor which is located directly above the sensing material 48. In another example, the optical measurement shown in FIG. 3B may be made after optical sensor assembly 36, restoring spring assembly 28, and microneedle 24 are withdrawn from the subject. In this case, sufficient time must elapse such that sensing material 48 integral to microneedle 24 undergoes enough of a color change to result in an accurate measurement.

The needle structures shown in FIGS. 1-3 can be very fine, on the order of a few to fifty microns in diameter at the tip. The fine geometries of 24 along with the relatively shallow penetration required to make the measurement significantly reduce the pain and discomfort to the subject. Another very significant issue for subjects requiring periodic glucose or other types of monitoring is compliance. Unfortunately many tests in the market such as the fingerprick test require the subject to take time away from their activities and make a measurement. Even other minimally-invasive prior art that use microneedles may require the subject to make the measurement. The embodiments described herein, and their equivalents, are uniquely advantaged in that they can be automated to perform periodic measurements without user intervention. As a result of its planar geometry, diagnostic systems can be made wearable having convenient, unobtrusive form factors and flat profiles.

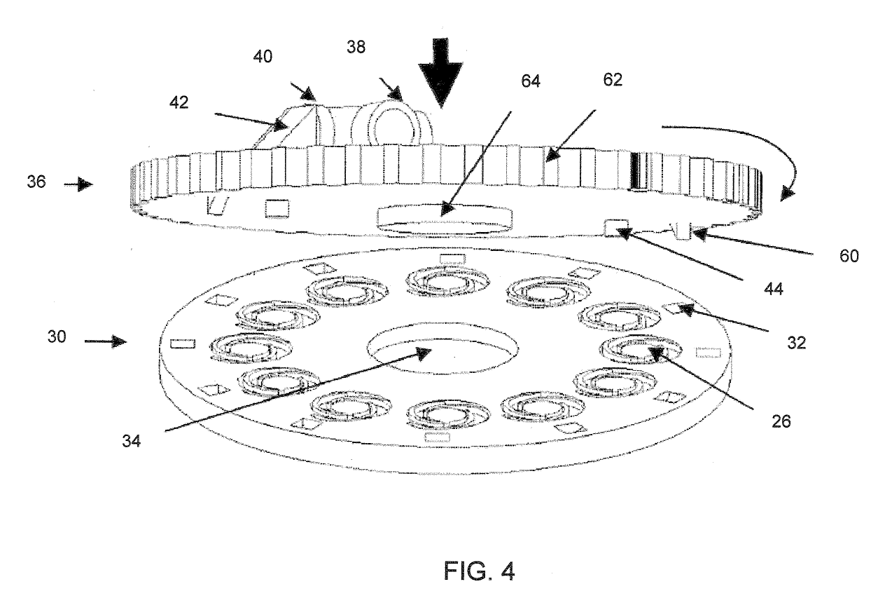

FIG. 4 illustrates an exploded view of one embodiment of an automatic blood monitor that incorporates microneedle array configuration 30 and optical sensor assembly 36. In this embodiment, optical sensor assembly 36 rotates concentrically relative to microneedle array configuration 30, revolving transparent depressors 44 over each microneedle base 26. Optical sensor assembly 36 is made to rotate around its axis using a motorized drive or other motion mechanism which may be meshed to a gearing mechanism 62 or any other rotary transport system. During this process, a spring or other biasing mechanism may be used to apply a force pushing the optical system 36 towards the microneedle array 30. Two, preferably three or more mechanical wedge spacers 60 are used to maintain a gap distance separating the microneedle array 30 and the optical system 36. Mechanical wedge spacers 60 are defined on the surface of the optical system assembly 36 such that when the transparent depressors 44 are located over microneedle bases 26, mechanical wedge spacers 60 all fall into positional encoder slots 32, pushing microneedle array 30 and optical system assembly 36 into close proximity. This action consequently forces the associated microneedle 24 toward the subject as shown in FIG. 3B. Mechanical wedge spacers 60 may have a, wedge-like geometry to activate the motion precisely, although other geometries may be used. Positional encoder slots 32 may also be shaped in a wedge-like geometry matching wedge spacers 60 and with a controlled slope, allowing mechanical wedge spacers 60 to rise out of mechanical encoder slots 32 as the optical system assembly 36 rotates. A radial alignment peg 64 concentric to the microneedle array 30 and optical system assembly 36 may be added to restrict lateral motion of the microneedle array 30 relative to the optical system assembly 36 during activation.

The number of mechanical encoder slots 32 relative to the number of microneedles maybe varied if needed. A 1:2 ratio in the number of microneedle:slot would result in only half of the needles being activated during a full rotation of the optical system assembly 36. This approach may be used for patients that require less number of measurements per interval of time. The same result may be achieved if the ratio is 1:1 and the rotational speed of the optical system assembly 36 is controlled.

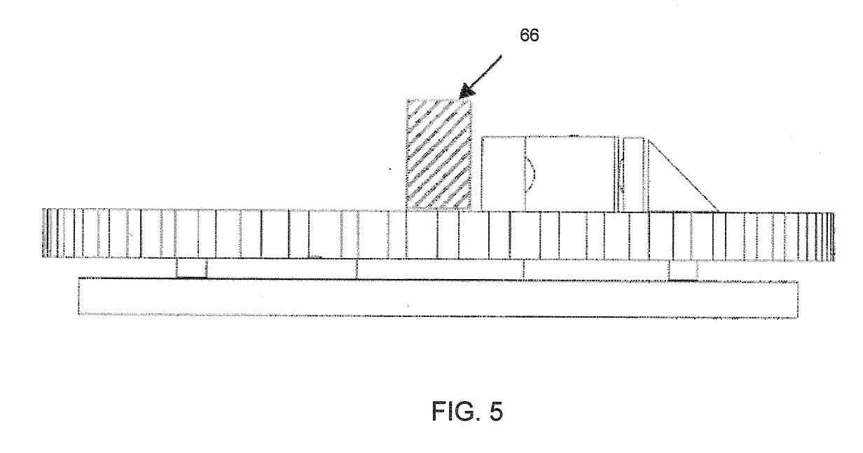

As mentioned previously, the invention provides a highly compact, programmable chemical monitoring system. FIG. 5 shows a side view of an embodiment of a biomedical monitor having a complete system configured for operation, including a biasing compression spring element 66 that provides a constant force on the optical system assembly 36 and microneedle array 30. The force applied by biasing spring 66 onto the optical system assembly 36 and microneedle array 30 needs to be sufficient to actuate the device (see FIG. 3B) and achieve insertion of the microneedles 24 into the subject. As a result of microfabrication methods and the efficient form factor of this design, full device dimensions could be highly compact, e.g. in the millimeter to centimeter range, although other dimensions may be used by those skilled in the art in order to meet various design goals. Microneedle array 30, due to its low cost, could be disposed of after the set of measurements is performed in accordance with the number of microneedles actuated. Although microneedle array 30 may be designed to monitor only one chemical such as glucose, different sensing materials 48 may be coated onto different microneedles, thereby providing the capability for more than one chemical to be monitored. In still other embodiments, more than one type of chemical sensing media may be coated onto a single microneedle, provided the multiple sensing media do not have conflicting spectral responses. In this manner, more than one chemical test could be performed at the same time with the same microneedle.

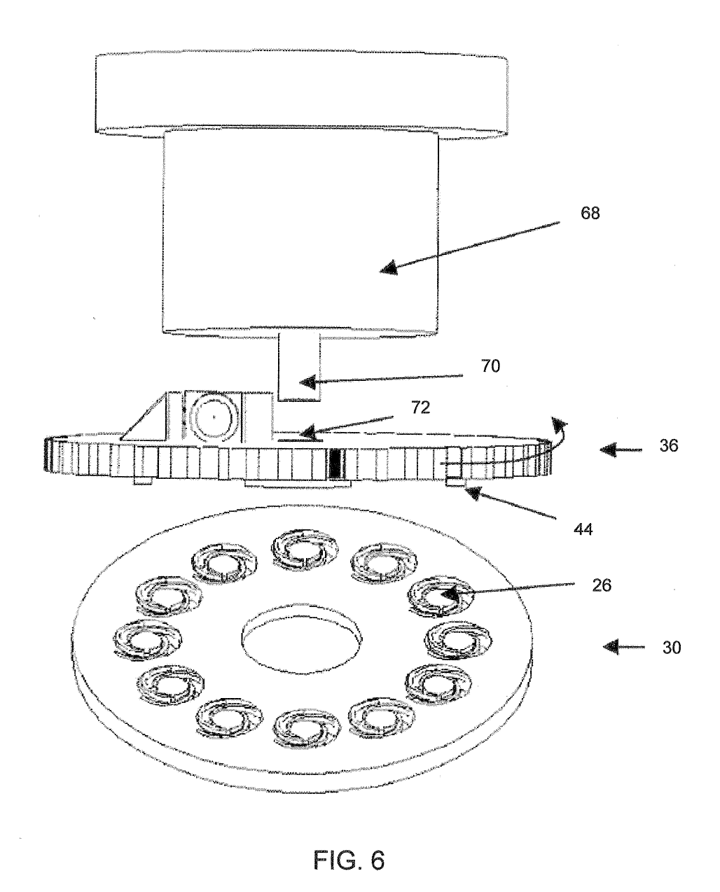

FIG. 6 illustrates an exploded view of another embodiment of a biomedical monitor that includes an electrically-controlled biasing device 68. In this example, electrically controlled biasing device 68 is activated causing moveable plunger 70 to apply pressure onto optical system assembly 36 and microneedle array 30. Given the additional degrees of control associated with electrically controlled biasing device 68, mechanical wedge spacers 60 and positional encoder slots 32 may not be required. In this configuration, the rate of revolution of optical system assembly 36 defines when activation can occur such that at least one transparent depressor 44 is aligned with a corresponding microneedle base 26. An optional cylindrical slot 72 may be used in optical system assembly 36 to restrict lateral motion of optical system assembly 36 relative to electronically controlled biasing device 68 and plunger 70.

The embodiments of biomedical monitors disclosed herein, and their equivalents have a variety of advantages which have been discussed throughout the specification. The embodied biomedical monitors may be attached to a subject and are able to make multiple sequential blood chemistry measurements. The biomedical monitor provides a highly useful device configuration and convenient fabrication process for dense arrays of individually actuated microneedles having integral sensors. The compact wearable device can sample body chemistry without extracting blood or interstitial fluid either during or after the microneedle is inserted in the subject. Consequently, the degree of invasiveness and risk of contamination is reduced, while improving the hygiene of the process. Due to their high multiplicity, microneedles with integral chemical sensing media may be inserted in the subject in sequence over an extended period of time, each chemical sensing element being required to make measurements for only a short time period. The use of each microneedle for a limited time may significantly reduce or eliminate the effect of bio-fouling. Sequential actuation of a multiple microneedles provides the ability for long term monitoring. Control of the serial actuation process can be programmed for a specific monitoring schedule, making the process more continuous and convenient for a subject. Due to their dense spacing and integrated actuation capability, many measurements may be made for extended time periods using a compact device worn by the subject as a small patch or chip. The biomedical monitor may be configured to sense chemicals which are naturally produced and/or found in a subject's body as well as chemicals which a subject has been exposed to, for example harmful toxins or biological components.

Having thus described several embodiments of the claimed invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. As just one example, it should be apparent that the biomedical monitor could be fabricated with individually addressable actuators for each microneedle, and individually readable image sensors for each microneedle such that neither the microneedle array nor the optical system would have to rotate. In such an embodiment, microsolenoids may be used for the individually addressable actuators. As one other non-limiting example, although rotational embodiments have been described herein, other embodiments may be translational in nature, such that the actuation motion is linear. Furthermore, the recited order of the processing elements or sequences, or the use of numbers, letters, or other designations therefore, is not intended to limit the claimed processes to any order except as may be specified in the claims. Accordingly, the claimed invention is limited only by the following claims and equivalents thereto.

What is claimed is:

1. A biomedical monitor, comprising:
an array of moveable microneedles coated with a first chemical sensing media;
an actuator configured to move at least one microneedle in the array of microneedles from a retracted position to an engaged position whereby the at least one microneedle enters a subject's skin;
an optical system configured to illuminate the at least one microneedle during or after entering the subject's skin and monitor the first chemical sensing media from the at least one microneedle, whereby at least one biomedical characteristic is determined based on at least one spectral property of the monitored first chemical sensing media.

2. The biomedical monitor of claim 1, wherein the array of microneedles comprise:
a substrate that defines a plurality of wells which house the microneedles in the array of moveable microneedles; and
one or more restoring spring elements coupled to each microneedle in the array of moveable microneedles.

3. The biomedical monitor of claim 2, wherein the one or more restoring spring elements are selected from the group consisting of a spiral spring, a cantilever spring, a flexible elastic membrane, and rubber.

4. The biomedical monitor of claim 2, wherein the substrate further comprises a material selected from the group consisting of silicon, silicon dioxide, silicon nitride, plastic, metal, glass, and dielectric material.

5. The biomedical monitor of claim 2, wherein the substrate further defines a cylindrical alignment slot for assisting in alignment of the array of microneedles with the actuator.

6. The biomedical monitor of claim 2, wherein the substrate further defines one or more positional encoder slots for assisting in alignment of the array of microneedles with the actuator.

7. The biomedical monitor of claim 6, wherein a ratio of positional encoder slots to microneedles is 1:1.

8. The biomedical monitor of claim 1, wherein the chemical sensing media comprises a media which changes color when in contact with a specific chemical specie.

9. The biomedical monitor of claim 1, wherein the chemical sensing media comprises a media which fluoresces when in contact with a specific chemical specie.

10. The biomedical monitor of claim 1, wherein the chemical sensing media comprises a material selected from the group consisting of glucose oxidase, glucose dehydrogenase, hexokinase-glucokinase, rhenium bipyridine, boronic acid having flourophores, NBD-fluorophores.

11. The biomedical monitor of claim 1, wherein the chemical sensing media comprises a polymeric matrix.

12. The biomedical monitor of claim 1, wherein the at least one biomedical characteristic is selected from the group consisting of cholesterol, HDL cholesterol, alcohol, estrogen-progesterone, cortisol, a physiological chemical, an ingested chemical, and an exposed chemical.

13. The biomedical monitor of claim 1, wherein the microneedles in the array of moveable microneedles are transparent.

14. The biomedical monitor of claim 1, wherein the microneedles in the array of moveable microneedles are translucent.

15. The biomedical monitor of claim 1, further comprising a second chemical sensing media, wherein at least one of the microneedles in the array of moveable microneedles is coated with the second chemical sensing media.

16. The biomedical monitor of claim 15, wherein the at least one microneedle coated with the second chemical sensing media is not coated with the first chemical sensing media.

17. The biomedical monitor of claim 15, wherein the at least one microneedle coated with the second chemical sensing media is also coated with the first chemical sensing media.

18. The biomedical monitor of claim 1, wherein the actuator comprises a plurality of individually addressable actuators which are configured to individually actuate each microneedle in the array of moveable microneedles.

19. The biomedical monitor of claim 18, wherein the individually addressable actuators comprise microsolenoids.

20. The biomedical monitor of claim 1, wherein the actuator comprises:
an actuation substrate which is configured to be rotated relative to the array of moveable microneedles;
a biasing device for biasing the actuation substrate towards the array of moveable microneedles; and
at least one depressor coupled to the actuation substrate for engaging at least one of the microneedles in the array of moveable microneedles using a force from the biasing device when the at least one depressor is aligned with the at least one microneedle.

21. The biomedical monitor of claim 20, wherein the biasing device comprises a solenoid.

22. The biomedical monitor of claim 20, wherein the biasing device is manually activated.

23. The biomedical monitor of claim 20, wherein the biasing device is a spring-loaded device.

24. The biomedical monitor of claim 20, wherein the actuation substrate comprises a toothed-surface for receiving rotational motion from a driven gear.

25. The biomedical monitor of claim 1, wherein the optical system comprises:
a light source configured to illuminate the at least one microneedle during or after entering the subject's skin; and
an image sensor configured to monitor the at least one spectral property of the first chemical sensing media.

26. The biomedical monitor of claim 25, wherein the image sensor is selected from the group consisting of: a CCD sensor, a multi-channel CCD sensor, a CMOS image sensor, a multi-channel CMOS image sensor, a spectrometer, a Bayer sensor, and a Foveon X3 sensor.

27. The biomedical monitor of claim 25, wherein the light source is selected from the group consisting of an incandescent light source, a light emitting diode, and a laser diode.

28. The biomedical monitor of claim 25, wherein the image sensor is oriented substantially over the at least one microneedle in the engaged position.

29. The biomedical monitor of claim 25, further comprising one or more optical elements to apply light from the light source to the at least one microneedle.

30. The biomedical monitor of claim 25, wherein the image sensor is configured to receive reflected light off of the first chemical sensing media from the light source.

31. The biomedical monitor of claim 25, wherein the image sensor is configured to receive diffuse light off of the first chemical sensing media from the light source.

32. The biomedical monitor of claim 1, wherein at least one microneedle in the microneedle array comprises a hollow needle.

33. The biomedical monitor of claim 1, wherein at least one microneedle in the microneedle array comprises a grooved needle.

34. The biomedical monitor of claim 1, wherein at least one microneedle in the microneedle array comprises a corrugated needle.

35. The biomedical monitor of claim 1, wherein the microneedle array comprises at least one needle of a first penetration depth and at least one needle of a second penetration depth which is different from the first penetration depth.

36. The biomedical monitor of claim 1, wherein the microneedle array comprises at least one needle with a cross-section that is selected from the group consisting of: square, rectangular, triangular, and circular.

37. The biomedical monitor of claim 1, wherein the microneedle array comprises at least one needle with a varying cross-section.

38. The biomedical monitor of claim 1, further comprising a film configured to separate the microneedles of the microneedle array from the subject's skin until each microneedle has been moved to the engaged position.

38. A replaceable array of moveable microneedles, comprising:
a plurality of microneedles coated with at least one chemical sensing media;
a substrate defining wells to house the microneedles; and
at least one restoring spring element coupled between each microneedle and the substrate such that each microneedle is held at least partially in an associated well.

39. The replaceable array of moveable microneedles according to claim 38, further comprising a film covering tips of the microneedles and the at least one chemical sensing media.

40. The replaceable array of moveable microneedles according to claim 38, wherein the substrate further defines a cylindrical alignment slot.

41. The replaceable array of moveable microneedles according to claim 38, wherein the substrate further defines one or more positional encoder slots.

42. A method of monitoring at least one biomedical characteristic, comprising:
engaging a first microneedle coated with a first chemical sensing media into a subject's skin;
illuminating the first chemical sensing media;
monitoring one or more spectral characteristics of light reflected from the first chemical sensing media; and
determining at least one biomedical characteristic based on the one or more spectral characteristics of light reflected from the first chemical sensing media.

43. The method of claim 43, further comprising:
waiting a desired period of time;
engaging a second microneedle coated with a second chemical sensing media into the subject's skin;
illuminating the second chemical sensing media;
monitoring one or more spectral characteristics of light reflected from the second chemical sensing media; and
determining at least one second biomedical characteristic based on the one or more spectral characteristics of light reflected from the second chemical sensing media.

44. The method of claim 43, wherein the first chemical sensing media and the second chemical sensing media comprise a same chemical sensing media.

45. The method of claim 43, wherein the at least one biomedical characteristic and the at least one second biomedical characteristic comprise a same biomedical characteristic.

46. The method of claim 43, further comprising, prior to engaging a second microneedle, withdrawing the first microneedle from the subject's skin.

47. The method of claim 42, wherein the at least one biomedical characteristic is selected from the group consisting of cholesterol, HDL cholesterol, alcohol, estrogen-progesterone, cortisol, a physiological chemical, an ingested chemical, and an exposed chemical.
